(19) United States
(12) Patent Application Publication
(10) Pub. No.: US 2016/0031770 A1
(43) Pub. Date: Feb. 4, 2016

(54) REACTOR WITH BAFFLE CONFIGURATION

(71) Applicant: Honeywell International Inc., Morristown, NJ (US)

(72) Inventors: Jing Guo, Colonial Heights, VA (US); Brendan T. Ackers, Wilmington, DE (US)

(21) Appl. No.: 14/790,112

(22) Filed: Jul. 2, 2015

Related U.S. Application Data
(60) Provisional application No. 62/030,222, filed on Jul. 29, 2014.

(51) Int. Cl. C07C 1/24; B01J 19/24; B01J 19/00

(52) U.S. Cl. CPC C07C 1/24; B01J 19/006; B01J 19/24; B01J 2219/0077; B01J 2219/24

(57) ABSTRACT

A reactor includes a shell defining an interior, a plurality of baffles positioned in the interior of the reactor, and a fluid pathway defined between the plurality of baffles and extending between an inlet and an outlet. In some embodiments, the reactor has a degree of mixing of less than 0.2.

### CROSS REFERENCE TO RELATED APPLICATION

This application claims the benefit under Title 35, U.S.C. §119(e) of U.S. Provisional Patent Application Ser. No. 62/030,222, entitled REACTOR WITH BAFFLE CONFIGURATION, filed on Jul. 29, 2014, the entire disclosure of which is expressly incorporated by reference herein.

### FIELD OF THE INVENTION

The present disclosure relates generally to reactor design, and more particularly to plug-flow type reactors.

### BACKGROUND

The dominant commercial method for producing phenol and acetone is by air oxidation of cumene to cumene hydroperoxide (CHP), followed by acid catalyzed decomposition of the CHP very selectively to phenol and acetone. Dimethylbenzyl alcohol (DMBA) is formed as the principle side product in the oxidation step and is subsequently dehydrated to alpha-methyl styrene (AMS) in a second acid catalyzed decomposition step. AMS is used commercially in the manufacture of plasticizers, resins and other polymers.

The dehydration reaction producing AMS is typically conducted in long tubes. In some situations, increasing the residence time of the reactants in the reactor may result in an improvement in the yield of AMS produced. However, to provide additional residence time in such a reactor would typically involve providing additional length to the reactor tube, which may require significant space.

Residence time of a reactor refers to the amount of time that a particular particle spends in the reactor. Mean residence time is generally defined as the volume of the reactor divided by the flow rate through the reactor. The residence time distribution of a reactor relates to the amount of time a particle is likely to spend in the reactor. The residence time distribution is a probabilistic function having a standard deviation about the mean residence time. The residence time distribution for a reactor is typically modeled based on either an ideal plug-flow reactor (PFR) or an ideal continuous-stirred tank reactor (CSTR). The degree of mixing of a reactor is a dimensionless value determined by dividing the variance of the residence time distribution by the square of the mean residence time.

In an ideal plug-flow reactor, fluid flowing through the reactor is conceptually viewed as a series of very thin sections, or “plugs.” As a plug flows through the reactor, there is assumed to be perfect mixing of the fluid in the radial direction within the plug (i.e., in a direction transverse to the flow direction), but no mixing of the fluid in the axial direction (i.e., forwards or backwards along the flow direction). Because there is no axial mixing, each element within the plug will have an identical residence time, and the standard deviation will be zero. The degree of mixing of a plug-flow reactor is theoretically 0.

In contrast, fluid in an ideal CSTR is assumed to be perfectly mixed throughout the reactor. Because each particle is assumed to have an equal probability of leaving the reactor at any given time, the standard deviation of the residence time distribution is high, and the degree of mixing of a CSTR is theoretically 1.

Actual reactors do not have residence time distributions of either ideal PFRs or CSTRs, but have a degree of mixing between 0 and 1. In some situations, it can be advantageous to maintain the degree of mixing near 0.

Improvements in the foregoing are desired.

### SUMMARY

The present disclosure provides a reactor having a high characteristic of plug flow distribution, while not requiring any liquid distribution device at the reactor inlet.

In some illustrative embodiments, the reactor provides a residence time distribution approaching or similar to a plug-flow reactor. In more particular embodiments, such a residence time distribution is achieved without a liquid distributor at the reactor inlet. In some illustrative embodiments, the reactor provides similar flow patterns at a wide variety of flow rates across a variety of design conditions. In some exemplary embodiments, the pressure loss through the reactor, due to flow direction change around baffles is within control limits of 7-8 kPa. In some exemplary embodiments including a gap between baffle edges and the interior shell of the reactor, the residence time distribution more closely resembles that of a plug-flow reactor, possibly due to observed leakage “shortcuts” observed with tracer material through such gaps.

In one exemplary embodiment, a reactor includes a shell defining an interior and a plurality of baffles positioned in the interior of the reactor. A fluid pathway extending between an inlet and an outlet of the reactor is defined between the plurality of baffles in the interior. In one more particular embodiment, the plurality of baffles comprises ten or more baffles, and a baffle cut for each baffle of the plurality of baffles is from 18% to 35%. In one more particular embodiment, the reactor has a degree of mixing less than 0.2.

In one more particular embodiment, the fluid pathway includes a plurality of changes in direction.

In another more particular embodiment of any of the above embodiments, the baffles are separated from the shell by at least one gap. In an even more particular embodiment, the gap has a width of about ½ inch or less.

In yet another more particular embodiment of any of the above embodiments, the inlet of the reactor does not include a liquid distributor.

In another exemplary embodiment, alpha-methyl styrene is produced from dimethylbenzyl alcohol by providing an inlet stream to an interior of a reactor, the inlet stream including dimethylbenzyl alcohol, wherein the reactor includes a plurality of baffles positioned in the interior of the reactor and the reactor has a degree of mixing of less than 0.2; and reacting at least a portion of the dimethylbenzyl alcohol in the reactor to form alpha-methyl styrene. In one more particular embodiment, the plurality of baffles comprises ten or more baffles, and a baffle cut for each baffle of the plurality of baffles is from 18% to 35%.

In one more particular embodiment, at least 75% of the dimethylbenzyl alcohol is reacted to form alpha-methyl styene.

In another more particular embodiment of any of the above embodiments, at least a portion of the dimethylbenzyl alcohol is passed through a gap between the baffle and at a wall defining the interior of the reactor, wherein the gap has a width of about ½ inch or less.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1A illustrates an exemplary reactor.

FIG. 1B illustrates a schematic view of an interior of the exemplary reactor of FIG. 1A.

FIG. 2 is a fragmentary view of a portion of the interior of the reactor of FIG. 1A including an exemplary set of baffles.

FIG. 3 illustrates a schematic view of an eleven baffle arrangement in an exemplary reactor in a vertical orientation.

FIG. 4 is a liquid phase velocity contour plot for the exemplary reactor of FIG. 3 in a vertical orientation with the inlet positioned above the outlet.

FIG. 5 is a liquid phase velocity contour plot for the exemplary reactor of FIG. 3 in a vertical orientation with the inlet positioned below the outlet.

FIG. 6 is a liquid phase velocity contour plot for the exemplary reactor of FIG. 3 in a horizontal orientation.

FIG. 7 illustrates a schematic view of a sixteen baffle arrangement in the exemplary reactor of FIG. 1A in a horizontal orientation.

FIG. 8 is a liquid phase velocity contour plot for the exemplary reactor of FIG. 7 in a horizontal orientation.

FIG. 9 illustrates a schematic view of a sixteen baffle arrangement in the exemplary reactor of FIG. 1A in a vertical orientation with the inlet positioned below the outlet.

FIG. 10 is a liquid phase velocity contour plot for the exemplary reactor of FIG. 9 with the inlet positioned below the outlet.

FIG. 11 is a liquid phase velocity contour plot for the exemplary reactor of FIG. 9 at a flow rate of 12,948 gal/hr (49,014 l/hr).

FIG. 12 is a liquid phase velocity contour plot for the exemplary reactor of FIG. 9 at a flow rate of 18,564 gal/hr (70,272 l/hr).

FIG. 13A illustrates the results of a tracer injection study in the exemplary reactor of FIG. 9 showing tracer distribution at 4 seconds following input at the inlet.

FIG. 13B illustrates the results of a tracer injection study in the exemplary reactor of FIG. 9 showing tracer distribution at 22 seconds following input at the inlet.

FIG. 13C illustrates the results of a tracer injection study in the exemplary reactor of FIG. 9 showing tracer distribution at 85 seconds following input at the inlet.

FIG. 14A illustrates the area weighted average for the tracer injection study.

FIG. 14B illustrates the degree of mixing of the exemplary reactor of FIG. 9 based on the tracer injection study.

FIG. 15A is a liquid phase velocity contour plot for the exemplary reactor of FIG. 9 including no spacing between the baffles and tank.

FIG. 15B is a liquid phase velocity contour plot for an exemplary cross section of FIG. 15A.

FIG. 16A is a liquid phase velocity contour plot for the exemplary reactor of FIG. 9 including spacing between the baffles and tank.

FIG. 16B is a liquid phase velocity contour plot for an exemplary cross section of FIG. 16A.

FIG. 17A illustrates the area weighted average for exemplary reactor of FIG. 16A including spacing between the baffles and tank.

FIG. 17B illustrates the degree of mixing of the exemplary reactor of FIG. 16A including spacing between the baffles and tank.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

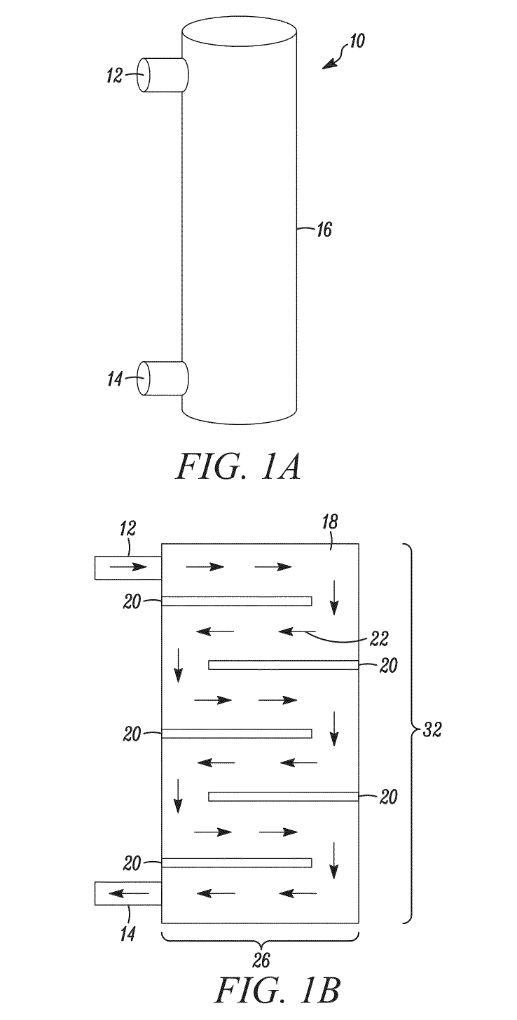

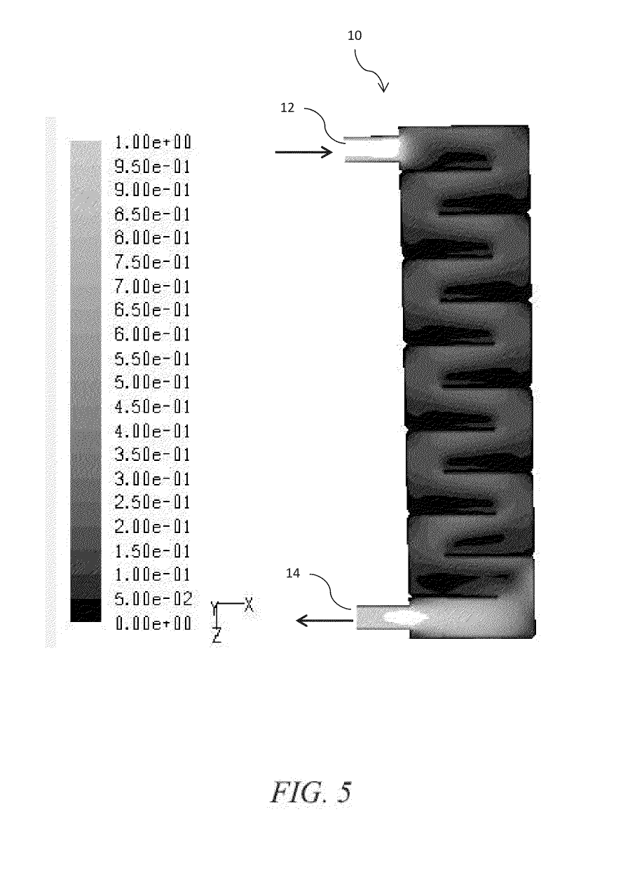

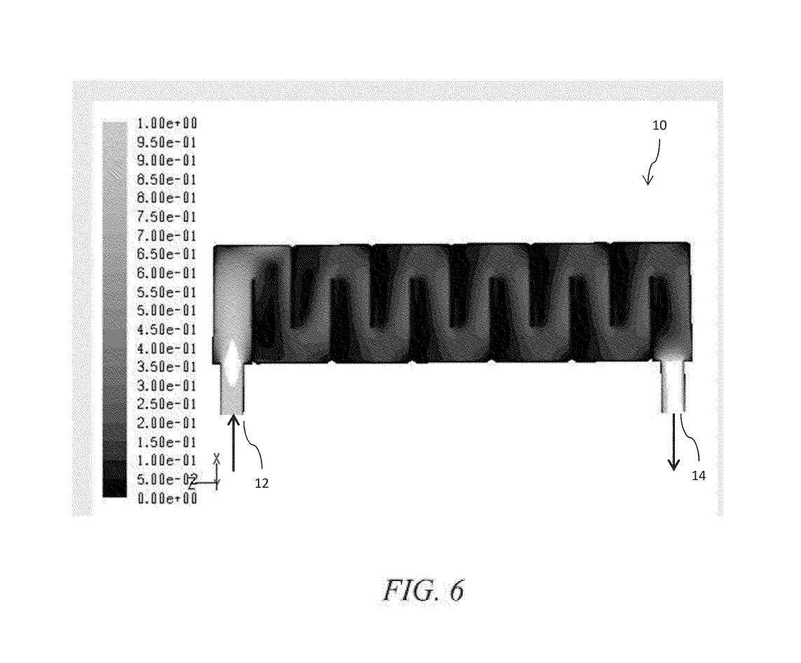

Referring first to FIG. 1A, an exemplary reactor 10 is illustrated. Reactor 10 includes an inlet 12 and an outlet 14. Although reactor 10 is illustratively shown in a vertical orientation with inlet 12 positioned above outlet 14, in other embodiments, outlet 14 may be positioned above inlet 12 (see FIG. 5), or reactor 10 may be in a horizontal orientation (see FIG. 6). The exterior shell 16 of reactor 10 encloses an interior 18.

Referring next to FIG. 1B, an exemplary interior 18 including a plurality of baffles 20 is illustrated. Interior 18 illustratively includes a flow path 22 fluidly connecting inlet 12 to outlet 14 of reactor 10. Baffles 20 interrupt the direct flow of the flow path 22 between inlet 12 and outlet 14.

In one illustrative embodiment, reactor 10 includes a plurality of baffles 20 positioned between inlet 12 and outlet 14. In some embodiments, reactor 10 may include as little as 10, 11, 12, 14, as many as 16, 18, 20, 22, or more baffles, or any range defined between any two of the foregoing values, such as 10 baffles to 22 baffles, 11 baffles to 20 baffles, or 12 baffles to 18 baffles.

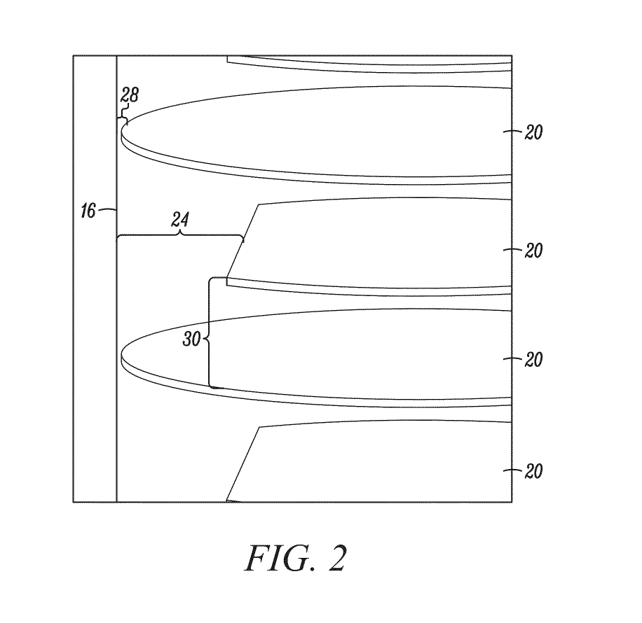

Referring next to FIG. 2, an illustrative position of a plurality of baffles 20 within the interior 18 of reactor 10 is shown. The spacing between adjacent baffles along an axial direction of reactor 10 defines a baffles spacing 30. In some embodiments, the baffle spacing as little as 3 inches, 4, inches, 5 inches, 6, inches, as great as 8 inches, 9 inches, 10 inches, 12 inches, or greater, or any range defined between any two of the foregoing values, such as 3 inches to 12 inches, 4 inches to 10 inches, or 6 inches to 9 inches.

Baffle cut refers to percentage of open area between the end of a given baffle 20 and the exterior shell 16. Baffle cut is calculated as the ratio of the distance 24 between the end of baffle 20 and the exterior shell 16 to the diameter 26 of reactor 10 (see FIG. 2). In some embodiments, the baffle cut may be as little as 18%, 20%, 23%, as great as 25%, 30%, 35%, or within any range defined between any two of the foregoing values, such as 18% to 35%, 20% to 30%, or 23% to 25%.

In some embodiments, the baffles 20 may be attached directly to the exterior shell 16 such that there is no circumferential gap 28 between baffle 20 and exterior shell 16 in addition to the primary gap provided by the baffle cut. In other embodiments, a circumferential gap 28 is present between baffle 20 and exterior shell 16. In one more particular embodiment, the circumferential gap is present around at least a portion of the circumference of each baffle 20. In one more particular embodiment, the circumferential gap is present around the entirety of the circumference of each baffle 20. Baffles 20 may be supported in position within the interior 18 of reactor 10 by one or more support structures (not shown), such as support structures coupling the baffles to one or more of the top or bottom of reactor 10 or exterior shell 16. In some embodiments, circumferential gap 28 is as little as ⅛ inch, 3/16 inch, ¼ inch, as great as 5/16 inch, ⅜ inch, ½ inch, or greater, or any value between any two of the foregoing values. In some embodiments, the inclusion of a non-zero gap reduces the degree of mixing within reactor 10, bringing reactor 10 closer to a theoretical plug-flow reactor.

In some embodiments, the reactor has a diameter 26 less than 4 inches, as little as 4 inches, 8 inches, 12 inches, 18 inches, 24 inches, as great as 30 inches, 36 inches, 42 inches, 48 inches, or greater, or within any range defined between any two of the foregoing values, such as 4 inches to 48 inches, 8 inches to 42 inches, or 24 inches to 36 inches.

In some embodiments, the reactor has a flow rate less than 1,000 gal/hr (3,785 l/hr), as little as 1,000 gal/hr (3,785 l/hr), 5,000 gal/hr (18,927 l/hr), 10,000 gal/hr (37,854 l/hr), 13,000 gal/hr (49,210 l/hr), as great as 15,000 gal/hr (56,781 l/hr), 20,000 gal/hr (75,708 l/hr), 25,000 gal/hr (94,635 l/hr), 30,000 gal/hr (113,562 l/hr), 40,000 gal/hr (151,416 l/hr), 50,000 gal/hr (189,271 l/hr), or greater, or within any range defined between any two of the foregoing values, such as 1,000 gal/hr (3,785 l/hr) to 50,000 gal/hr (189,271 l/hr), 5,000 gal/hr (18,927 l/hr) to 30,000 gal/hr (113,562 l/hr), or 10,000 gal/hr (37,854 l/hr) to 25,000 gal/hr (94,635 l/hr).

A residence time distribution (RTD) curve can be used to determine a mean residence time and a the mixing degree. Residence time of a reactor refers to the amount of time that a particular particle spends in the reactor. The average residence time is given by the first moment of the age distribution:

t̅=∫₀^{∞}t·E(t)dt

In some embodiments, the reactor 10 has a mean residence time as little as 50 seconds, 60 seconds, 70 seconds, 80 seconds, 85 seconds, as great as 90 seconds, 100 seconds, 110 seconds, 120 seconds, 130 seconds, or greater, or within any range defined between any two of the foregoing values, such as 50 seconds to 130 seconds, 60 seconds to 120 seconds, or 80 seconds to 100 seconds.

The second central moment indicates the variance (σ²), the degree of dispersion around the mean:

σ²=∫₀^{∞}(t− t̅)²·E(t)dt

The degree of mixing is the dimensionless ratio of the variance to square of the mean residence time:

$${δ}_{θ}^{2} = \frac{{σ}^{2}}{{\overline{t}}^{2}}$$

In some embodiments, the reactor 10 has a degree of mixing approaching that of a theoretical plug-flow reactor. In some embodiments, the degree of mixing is as little as 0.3, 0.2, 0.15, 0.1, 0.09, 0.08, or less, or within any range defined between any two of the foregoing values, such as 0.3 to less than 0.08, 0.2 to less than 0.08, or 0.15 to 0.08.

In some embodiments, the reactor 10 does not include a liquid distributor in the inlet 12. Liquid distributors are typically used in reactor columns to provide uniform liquid distribution within the reactor. However, plugging or fouling of the liquid distributor may occur at the distributor opening area. In some embodiments, the likelihood of plugging or fouling within the reactor is reduced or eliminated by not including a liquid distributor. Additionally, the space between the inlet 12 and any distributor within the interior 18 of reactor 10 may consume valuable reactor volume, increasing the necessary size of reactor 10. In some embodiments, the size of the reactor 10 is reduced by not including a liquid distributor. In some embodiments, a reactor 10 without a liquid distributor provides a low pressure head loss, a wide operating range of conditions, and increased utilization of the interior 18 of reactor 10 for performing a reaction.

In one illustrative embodiment, the inlet 12 of reactor 10 includes dimethylbenzyl alcohol, and at least a portion of the dimethylbenzyl alcohol is reacted in the interior 18 of reactor 10 to form alpha-methyl styrene. In some embodiments, the degree of conversion of dimethylbenzyl alcohol to alpha-methyl styrene is as little as 50%, 60%, 70%, 75%, 80%, as great as 90%, 95%, 98%, 99%, 99.5%, or greater, or between any range defined between any two of the foregoing values, such as 50% to 99.5%, 60% to 99%, or 80% to 95%.

In a more particular embodiment, the inlet 12 of reactor 10 includes a first inlet flow composition, comprising dimethylbenzyl alcohol. In some embodiments, the first inlet flow composition comprises a weight percentage of dimethylbenzyl alcohol, based on the total weight of the first inlet flow composition, from as little as 0.5 wt. %, 1 wt. %, 2 wt. % 2.5 wt. %, 3 wt. %, as great as 4 wt. %, 5 wt. %, 10 wt. %, 20 wt. %, or greater, or between any range defined between any two of the foregoing values, such as 0.5 wt. % to 20 wt. %, 1 wt. % to 10 wt. %, or 2 wt. % to 10 wt. %. In some embodiments, the first inlet flow composition comprises a weight percentage of water, based on the total weight of the first inlet flow composition, from as little as 0 wt. %, 0.5 wt. %, 1 wt. %, 1.5 wt. %, as great as 2 wt. %, 2.5 wt. %, 3 wt. %, 5 wt. %, or greater, or between any range defined between any two of the foregoing values, such as 0.5 wt. % to 5 wt., 1 wt. % to 3 wt. %, or 1 wt. % to 2 wt. %. In some embodiments, the first inlet flow composition optionally includes at least one of cumene, cumene hydroperoxide, phenol, or acetone.

### EXAMPLES

### Example 1

### Effect of Reactor Orientation

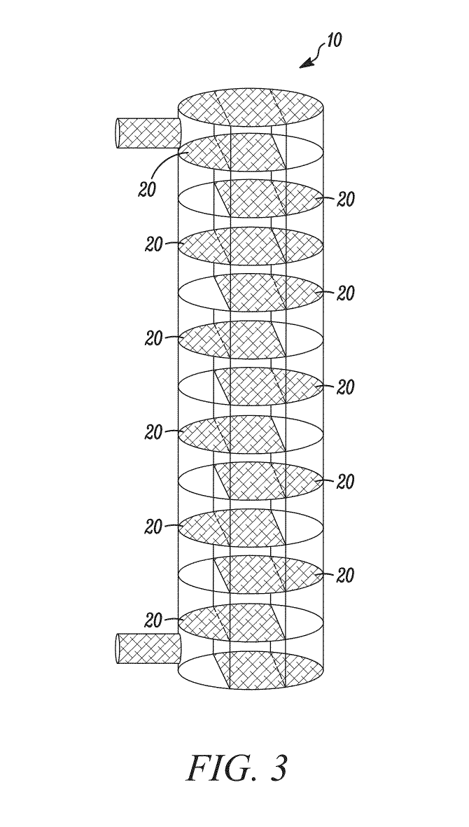

Referring next to FIGS. 3-6, the effect of the orientation of reactor 10 on fluid dynamics was investigated. Fluid dynamics were determined using ANSYS Fluent computational fluid dynamics (CFD) simulation software available from ANSYS Inc., Cannonsburg, Pa.

An exemplary reactor 10 containing eleven baffles 20 is illustrated in FIG. 3. Baffle spacing was set to 10 inches with a baffle cut of 30%, and a target residence time of 85 seconds. The reactor total length 32 (see FIG. 1B) was 120 inches.

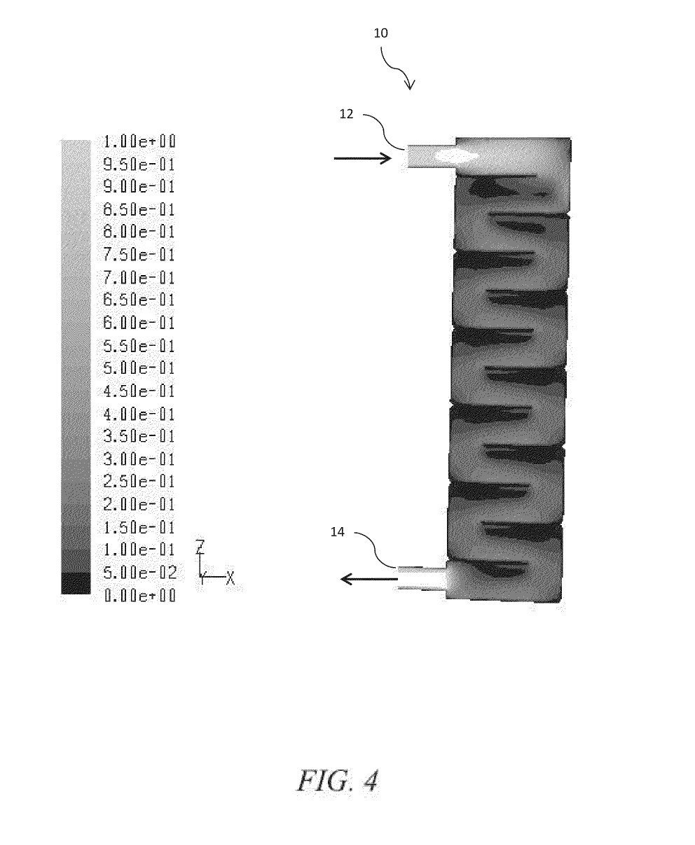

A liquid phase velocity contour plot for each of three orientations is provided in FIGS. 4-6. In FIG. 4, the reactor 10 was oriented vertically and inlet 12 is positioned above outlet 14, and liquid flows downwardly around the baffles 20 in the interior 18 of reactor 10 to the outlet 14. In FIG. 5, the reactor 10 is oriented vertically and inlet 12 is positioned below outlet 14, and liquid flow is forced upwardly through the interior 18 of reactor 10 around the baffles 20. In FIG. 6, the reactor 10 is positioned horizontally with liquid injected from an inlet 12 in the tank bottom.

For the liquid velocity distributions, such as illustrated in FIGS. 4-6, the liquid velocity at each point in the reactor is indicated by the color of that point. A gray scale for each Figure is provided, with relatively low velocities indicated by black, and relatively high velocities indicated by white.

As shown in FIGS. 4-6, the liquid velocity distribution results for each of the three orientations is substantially similar. While not wishing to be bound by any theory, these results suggest that the number, size, and position of baffles 20 are dominant factors to affect the liquid velocity distribution within the reactor 10. A comparison of FIGS. 4 and 5 indicates that liquid upflow leads to higher axial dispersion compared to liquid downflow, but the shape of the residence time distribution curve and plug-flow characteristic of the reactor 10 would be controlled primarily by the spacing and size of baffles 20.

### Example 2

### Effect of Baffle Parameters

Example 1 investigated a reactor 10 including eleven baffles. The effect of the number and spacing of baffles was further investigated.

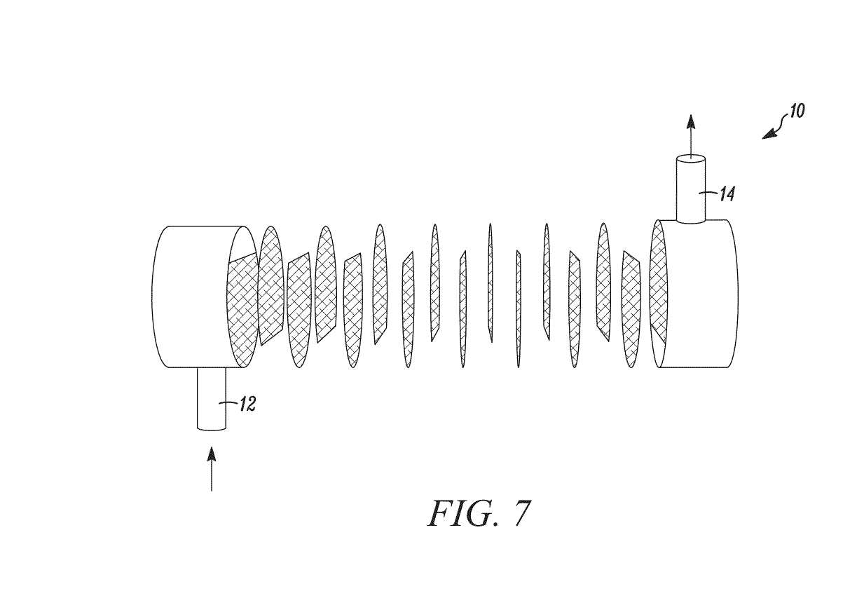

An exemplary reactor 10 including sixteen baffles 20 was evaluated using the CFD simulation, as shown in FIG. 7. Baffle spacing was set to 6 inches with a baffle cut of 23%. Because an even number of baffles 20 were included, the outlet 14 of the reactor 10 was opposite that shown in FIG. 6, in which an odd number of baffles were included. The reactor 10 was otherwise unchanged from that of FIG. 6.

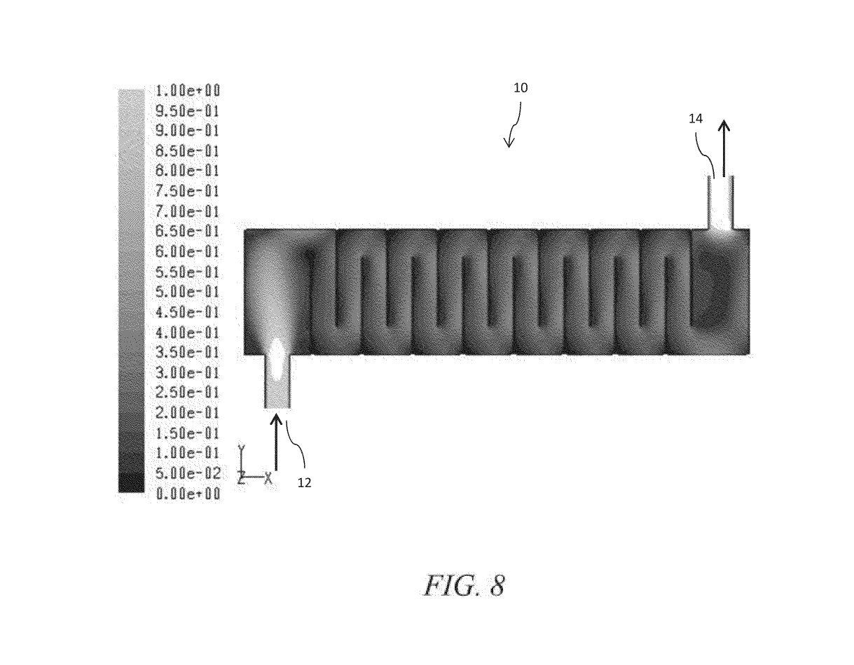

A liquid phase velocity contour plot for the reactor 10 of FIG. 7 is provided in FIG. 8. Compared to the eleven baffle reactor 10 of Example 1 (FIGS. 4-6), the sixteen baffle reactor 10 as shown in FIG. 8 exhibited less dead zone volume and more uniform velocity distribution. These results suggest a flow type characteristic approximating a plug-flow reactor.

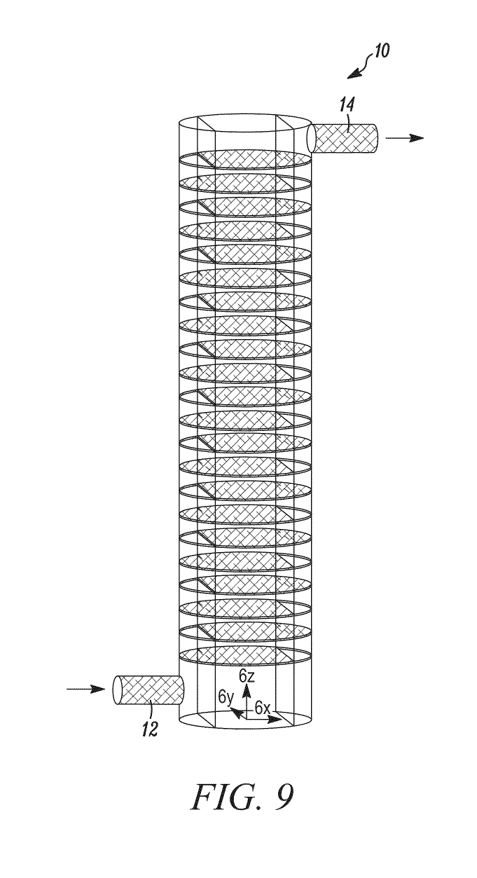

Referring next to FIG. 9, an exemplary reactor 10 including twenty-two baffles 20 was evaluated using the CFD simulation, as shown in FIG. 9. Baffle spacing was set to 5 inches with a baffle cut of 20%. Because an even number of baffles 20 were included, the outlet 14 of the reactor 10 was opposite that shown in FIG. 6, in which an odd number of baffles were included. The reactor total length 32 (see FIG. 1B) was 136 inches. The reactor 10 was otherwise unchanged from that of FIG. 5.

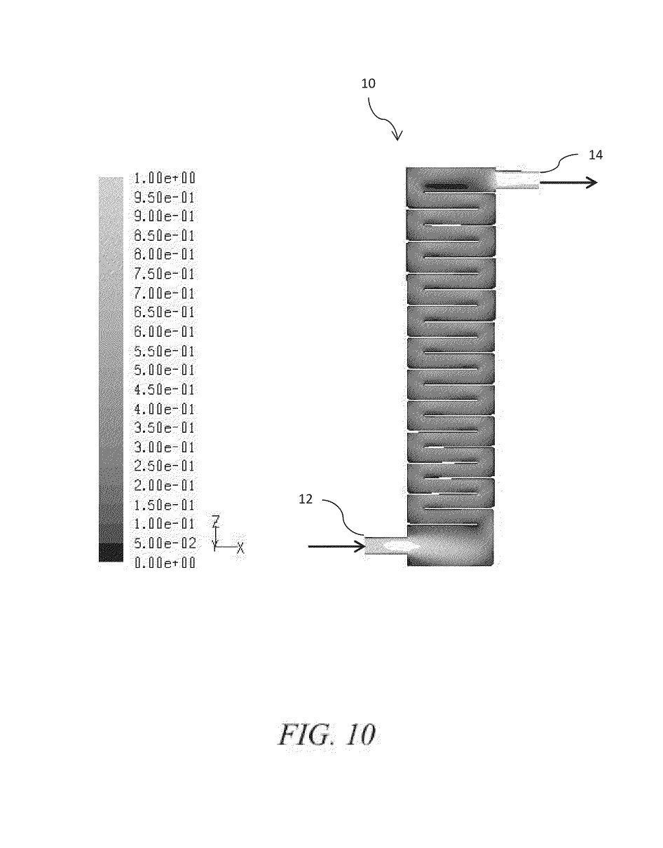

A liquid phase velocity contour plot for the reactor 10 of FIG. 9 is provided in FIG. 10. Compared to the sixteen baffle reactor 10 (FIGS. 7-8), the twenty-two baffle reactor 10 as shown in FIG. 9 exhibited less dead zone volume. Due to the smaller baffle cut, resulting in a smaller open area, and inclusion of additional baffles, the pressure head loss increased from 160 Pa/baffle for the twenty-two baffle reactor 10 of FIG. 9, compared to 149 Pa/baffle of the sixteen baffle reactor of FIG. 7. However, this increase was relatively small in size.

The presence of additional baffles led to a longer flow path 22 from inlet 12 to outlet 14 (see FIG. 1B), but smaller baffle spacing provides a higher flow velocity passing through void space. The combination of longer flow path 22 and higher velocity combined to provide a similar residence time, and thus a similar reaction conversion rate.

### Example 3

### Capacity Study

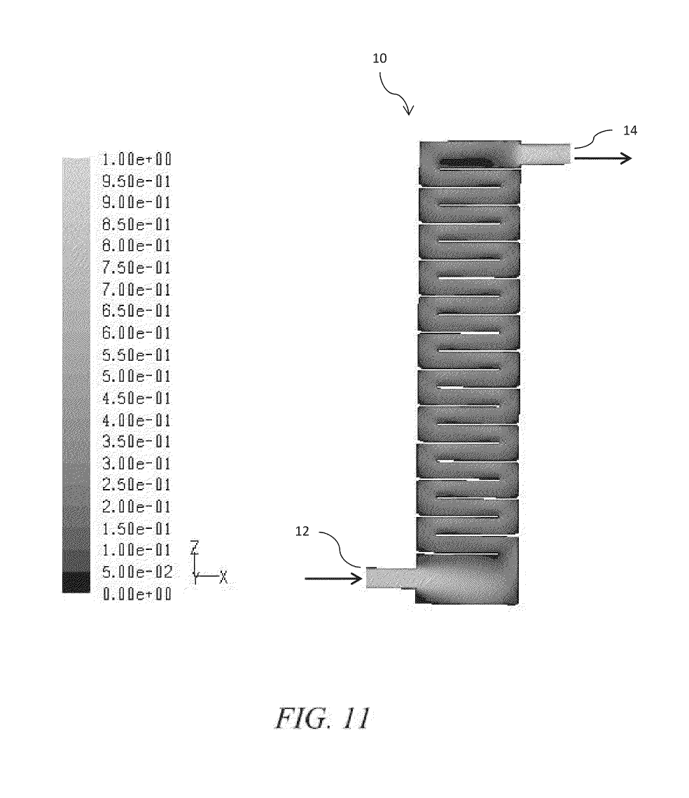

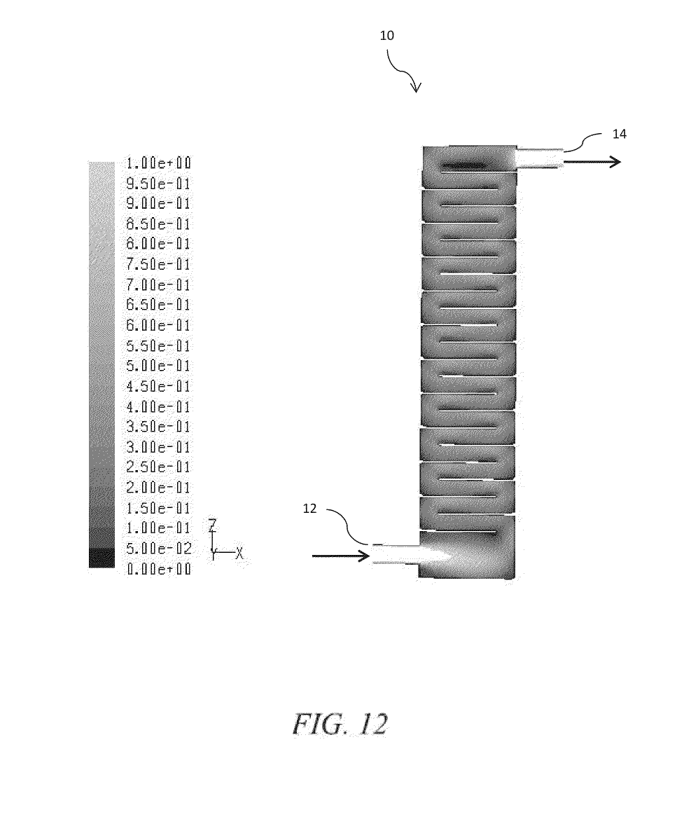

The effects of various flow rates on the twenty-two stage reactor 10 of FIG. 9 were investigated. The liquid phase velocity contour plot of FIG. 10 reflects a nominal flowrate of 16,969 gal/hr (64,235 l/hr). The CFD simulation was used to generate similar liquid phase velocity contour plots for a low flowrate value of 12,948 gal/hr (49,014 l/hr) (FIG. 11) and a high flowrate value of 18,564 gal/hr (70,272 l/hr) (FIG. 12). The legends for FIGS. 11 and 12 are kept the same for visual comparison. Within the low and high tested values, it appears that plug flow type can be attained within reactor 10, regardless of the particular flowrate.

### Example 4

### Residence Time Distribution Study

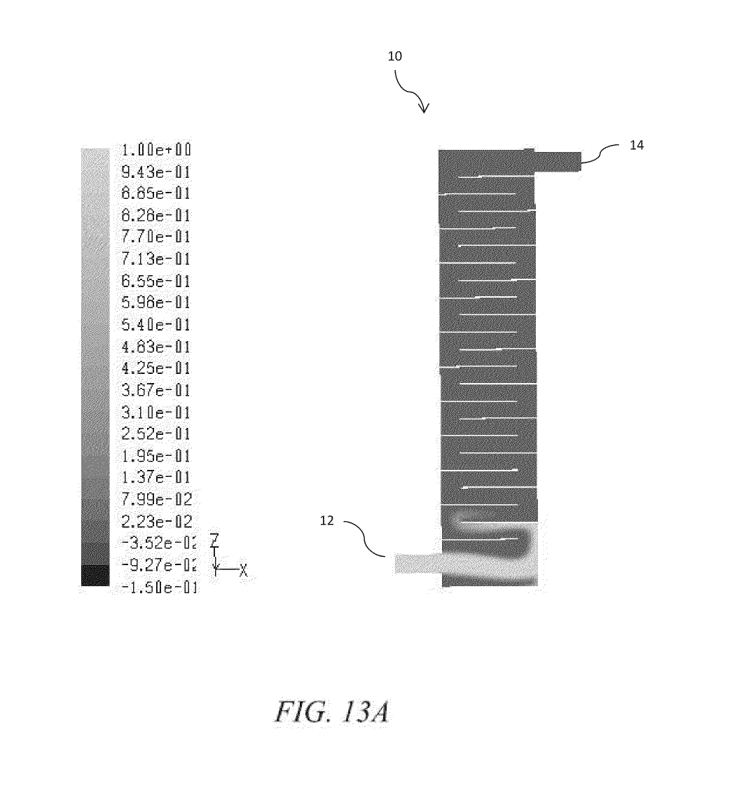

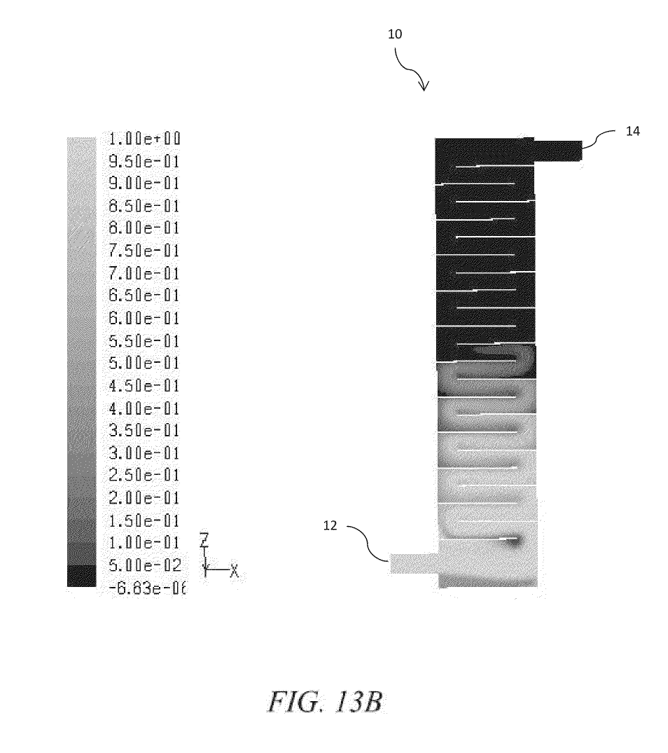

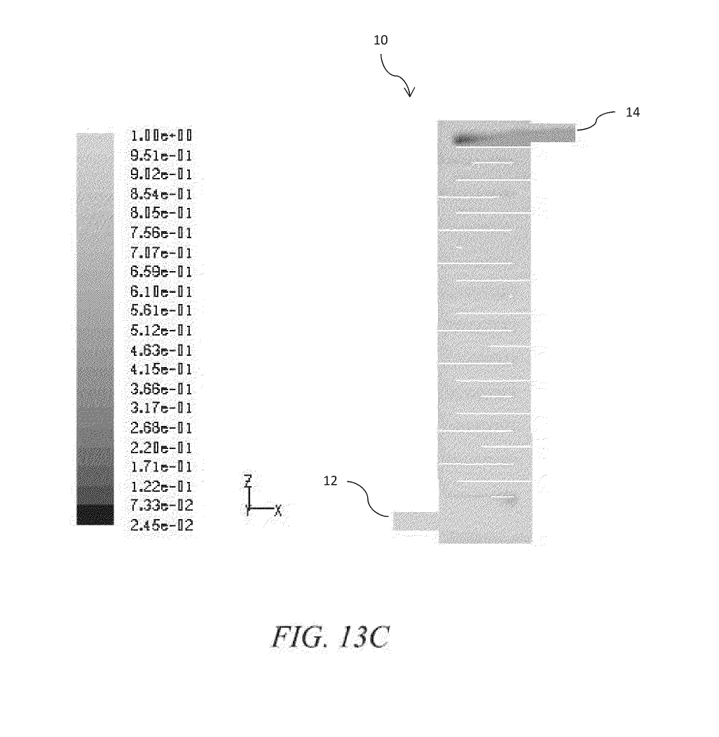

The plug-flow characteristic of the twenty-two stage reactor 10 of FIG. 9 was investigated using a simulated tracer injection study with the CFD simulation. FIGS. 13A-13C illustrate the presence of tracer at various times following input of the tracer at the inlet 12 of the reactor 10. For each of FIGS. 13A-13C, the concentration of the tracer at each point in the reactor is indicated by the color of that point. A color scale for each Figure is provided, with relatively low concentrations indicated by blue, and relatively high concentrations indicated by red. The color black indicates baffle geometry and shape.

FIG. 13A shows the tracer distribution at 4 seconds following input. FIG. 13B shows the tracer distribution at 22 seconds following input. FIG. 13C shows the tracer distribution at 85 seconds following input.

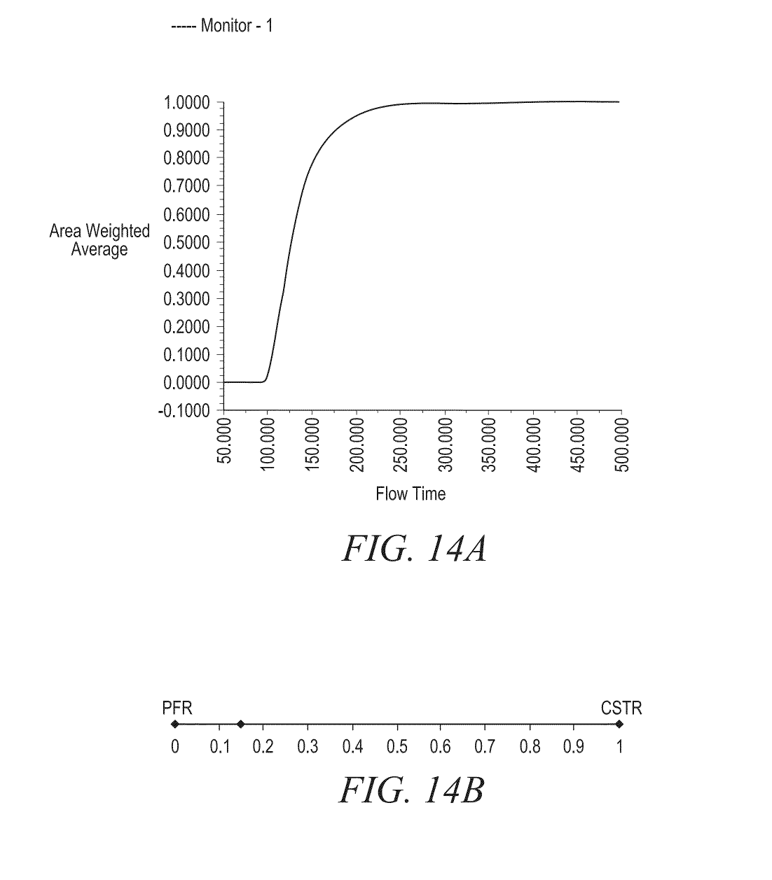

The residence time distribution (RTD) curve provided in FIG. 14A was determined from the tracer study. A first moment of the RTD is determined for mean residence time, and a second moment is determined for mixing degree. The average residence time is given by the first moment of the age distribution:

t̅=∫₀^{∞}t·E(t)dt

The second central moment indicates the variance (σ²), the degree of dispersion around the mean:

σ²=∫₀^{∞}(t− t̅)²·E(t)dt

The degree of mixing is the dimensionless ratio of the variance to square of the mean residence time:

$${δ}_{θ}^{2} = \frac{{σ}^{2}}{{\overline{t}}^{2}}$$

In the twenty-two baffle reactor 10, which includes no liquid distributor, flow direction change and dead zones exist around the baffles. This leads to a certain level of back mixing within the reactor 10. However, based on the RTD measurement, the calculated mixing degree is δ²ₑ=0.148, and the flow type approaches that of a plug-flow reactor, as shown in FIG. 14B.

### Example 5

### Effect of Circumferential Gaps Between Baffle and Shell

In a typical reactor 10, the baffle 20 assembly is designed to be pullable, or removable. This results in a circumferential gap 28 between the edge of the baffles 20 and the shell 16. It was desired to determine the effect of a small circumferential gap 28 on the mixing degree using the CFD simulation.

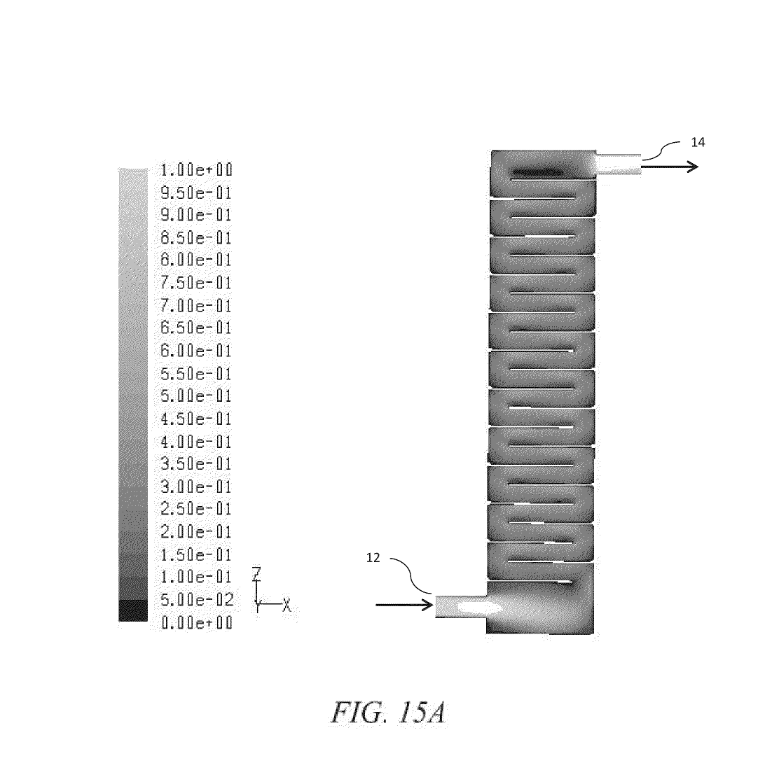

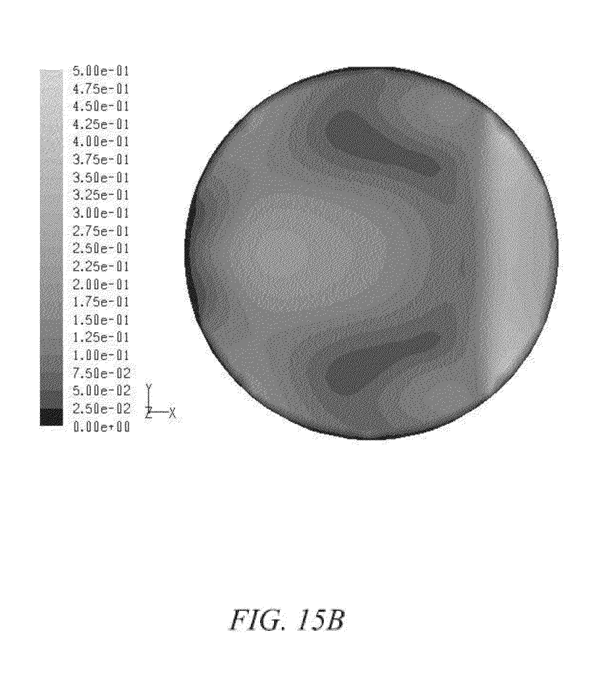

The liquid phase velocity contour plot of FIG. 15A reflects the twenty-two baffle reactor 10 as shown in FIG. 9 without the inclusion of the circumferential gap 28. The liquid phase velocity plot shown in FIG. 15B is a top view taken at an elevation of 3.13 meters in the reactor 10.

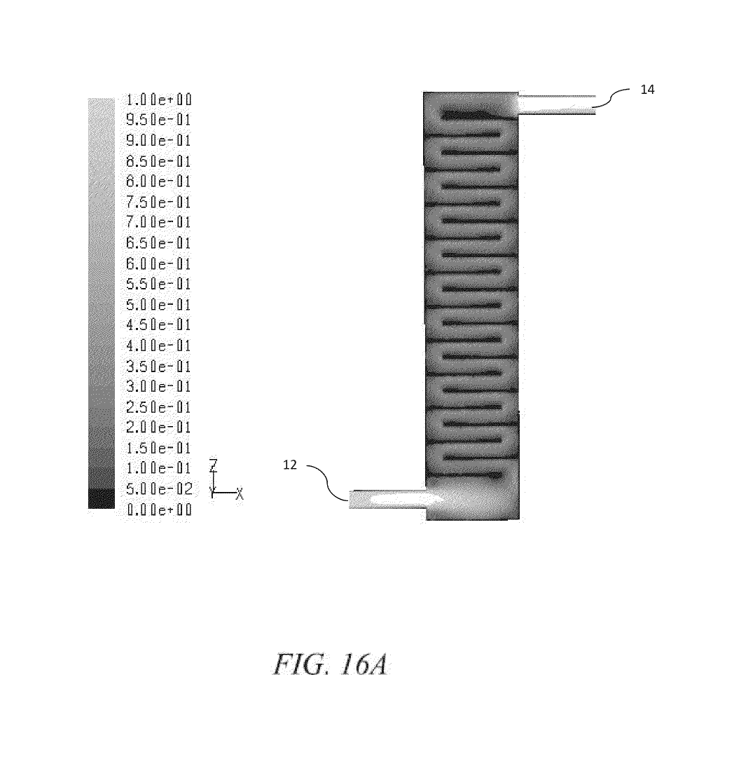

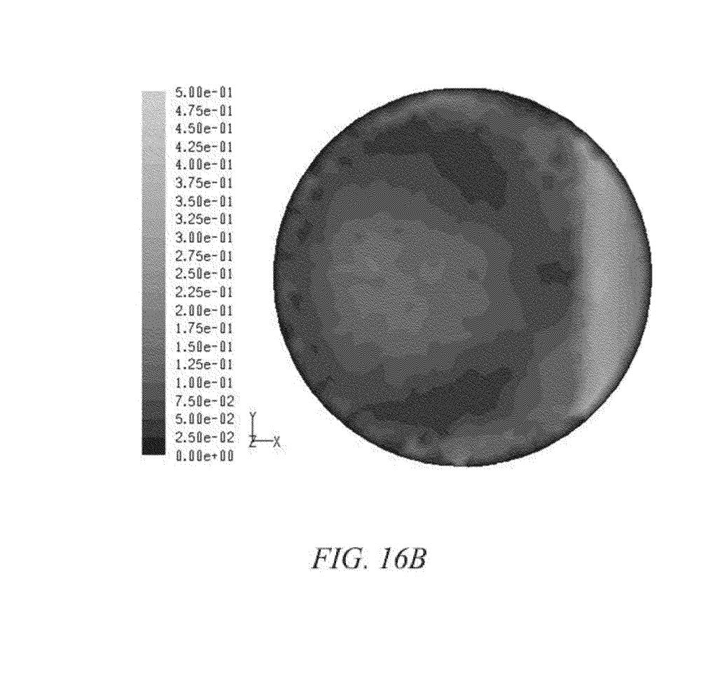

The liquid phase velocity contour plot of FIG. 16A reflects the twenty-two baffle reactor 10 as shown in FIG. 9 with the inclusion of a 3/16 inch circumferential gap 28 between the baffle 20 and shell 16. The liquid phase velocity plot shown in FIG. 16B is a top view taken at an elevation of 3.13 meters in the reactor 10.

As can be seen in FIGS. 15 and 16, visible flow distribution difference exists between these two cases. The inclusion of the circumferential gap 28 reduced the amount of less dead zone volume in the reactor 10.

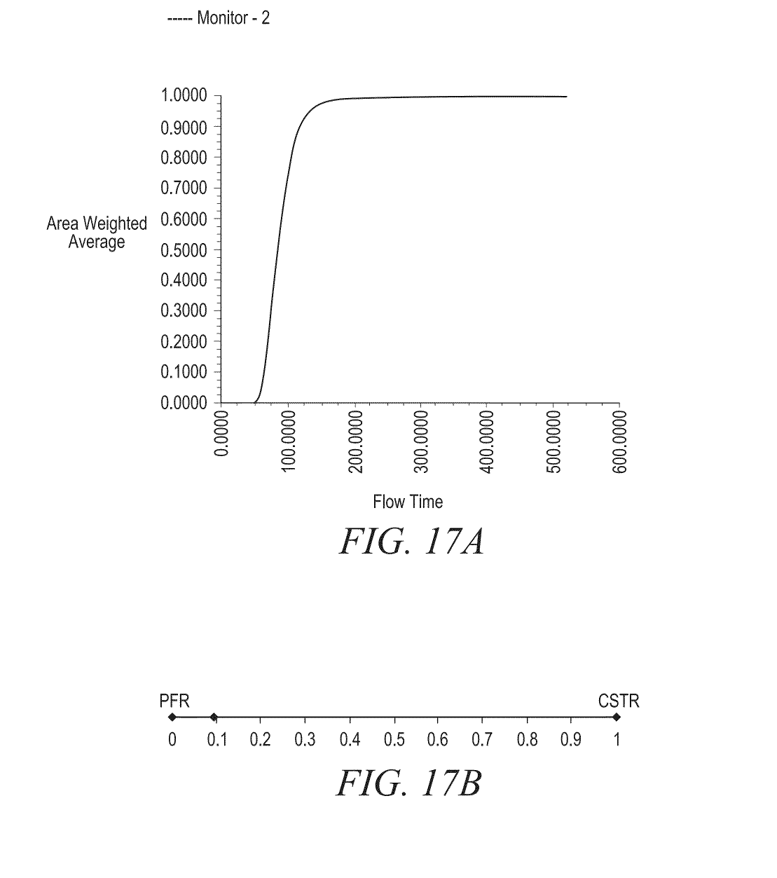

A tracer injection study was conducted for the reactor 10 including the 3/16 inch gap illustrated in FIG. 16. The RTD curve is presented in FIG. 17A. In comparison with Example 4, which did not include a circumferential gap 28, the 3/16 inch gap reduced the RTD variance. Without wishing to be bound by any theory, it is believed that the inclusion of the gap allowed a portion of the fluid to flow between the baffle and the shell rather than around the full length of the baffles. This flow reduced the amount of dead zone and slightly reduced the mixing degree, bringing the reactor closer to a theoretical plug-flow reactor, as shown in FIG. 17B. Compared to Example 4, the reactor 10 including the gap, which accounted to 1.78% of total reactor cross sectional area, the standard deviation of the residence time distribution was slightly lower, decreasing the mixing degree to δ²ₑ=0.088, and bringing the reactor type a bit closer to PFR, as seen in FIG. 17B.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A reactor comprising:
a shell defining an interior;
a plurality of baffles positioned in the interior of the reactor, wherein the plurality of baffles comprises ten or more baffles, and a baffle cut for each baffle of the plurality of baffles is from 18% to 35%; and
a fluid pathway defined between the plurality of baffles and extending between an inlet and an outlet;
wherein the reactor has a degree of mixing of less than 0.2.

2. The reactor of claim 1, wherein the fluid pathway includes a plurality of changes in direction.

3. The reactor of claim 1, wherein the plurality of baffles comprises from 10 to 22 baffles.

4. The reactor of claim 1, wherein the baffles are separated from the shell by at least one gap.

5. The reactor of claim 4, wherein the gap has a width of about ½ inch or less.

6. The reactor of claim 1, wherein each baffle of the plurality of baffles is attached directly to the shell such that there is no circumferential gap between the baffle and the shell.

7. The reactor of claim 1, wherein the reactor has a mean residence time of 50 seconds to 130 seconds.

8. The reactor of claim 1, wherein the reactor has a degree of mixing of less than 0.1.

9. The reactor of claim 1, wherein the reactor does not include a liquid distributor.

10. A method of producing alpha-methyl styrene from dimethylbenzyl alcohol comprising:
providing an inlet stream to an interior of a reactor, the inlet stream including dimethylbenzyl alcohol, wherein the reactor includes a plurality of baffles positioned in the interior of the reactor, wherein the plurality of baffles comprises ten or more baffles, a baffle cut for each baffle of the plurality of baffles is from 18% to 35%, and the reactor has a degree of mixing of less than 0.2; and
reacting at least a portion of the dimethylbenzyl alcohol in the reactor to form alpha-methyl styrene.

11. The method of claim 10, wherein at least 75% of the dimethylbenzyl alcohol is reacted to form alpha-methyl styene.

12. The method of claim 10, further comprising passing at least a portion of the dimethylbenzyl alcohol through a gap between the baffle and at a wall defining the interior of the reactor, wherein the gap has a width of about ½ inch or less.

13. The method of claim 10, wherein the plurality of baffles comprises from 10 to 22 baffles

14. The method of claim 10, wherein the reactor has a diameter from 4 inches to 48 inches.

15. The method of claim 10, wherein the reactor has a flow rate of 1,000 gal/hr to 50,000 gal/hr.

16. The method of claim 10, wherein the reactor has a mean residence time of 50 seconds to 130 seconds.

17. The method of claim 10, wherein the reactor has a degree of mixing of less than 0.1.

18. The method of claim 10, wherein the reactor does not include a liquid distributor.

19. The method of claim 10, wherein the inlet stream comprises from 0.5 to 20 wt. % dimethylbenzylalcohol, based on a total weight of the inlet stream composition.

20. The method of claim 10, wherein the inlet stream comprises at least one of cumene, cumene hydroperoxide, phenol, and acetone.
